(19) United States
(12) Patent Application Publication
(10) Pub. No.: US 2005/0042187 A1
(43) Pub. Date: Feb. 24, 2005

(54) High skin friction cosmetic creams containing dispersed zinc oxide particles as inorganic sunscreen

(75) Inventors: Ritu Verma, New York, NY (US); Yonghui Yuan, Tappan, NY (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/645,576

(22) Filed: Aug. 21, 2003

(51) Int. Cl. A61K 7/42; A61K 7/021

(52) U.S. Cl. 424/59; 424/63

(57) ABSTRACT

A high skin friction cosmetic composition that can provide the consumer-desired sensory properties of traditional vanishing creams, containing solid asymmetric particles and ZnO incorporated by an inventive process. According to the inventive process the temperature during the mixing of ZnO and asymmetric particles is controlled at less than 80° C. and the time of mixing is limited to about 10 minutes. After about 10 minutes, the reaction is quenched by cooling to about 50° C.

### FIELD OF THE INVENTION

The invention relates to cosmetic skin creams providing a high skin friction and the dispersion therein of zinc oxide particles used as inorganic sunscreen.

### BACKGROUND OF THE INVENTION

Consumers living in hot, humid climates, or consumers with oily skin, desire cosmetic products that have unique tactile properties during use.

Specifically, such products should, upon application to the skin, deliver a high skin friction and a matte finish to overcome the oily skin feel and shiny skin appearance. The greater the increase in skin friction, the less greasy the user perceives the product to be. See Laufer et al., Objective Measurement and Self-Assessment of Skin-Care Treatments, Cosmetics and Toiletries Magazine, Vol. 111, Jun. 1996, pp. 92-96.

More specifically, the typical sensory and optical features of these high skin friction and matte finish creams, also known as vanishing creams, are: 1) provide a dry, draggy, non-greasy feel to skin; 2) provide a non-shiny, matte finish; 3) spread easily on the skin; 4) absorb or “vanish” rapidly into the skin. The traditionally used vanishing creams contain high levels of stearic acid and alkaline metal soap as an emulsifier, which is important to physical stability of the product. The soap is formed by the in situ neutralization with caustic potash or other alkali on a portion of the stearic acid, i.e. the creams are made by saponification of stearic acid. Vanishing creams are disclosed in Bartolone et al., U.S. Pat. No. 6,153,177.

Sunscreens are a desirable inclusion in vanishing creams. Inorganic sunscreens have an advantage over organic sunscreen due to higher permitted levels in cosmetic compositions, of up to about 25%, as opposed to only several percent for organic sunscreens. Zinc oxide (ZnO) particles are advantageous as inorganic sunscreen in the personal care industry because of its broad ultraviolet (UV) absorbance spectrum and non-toxic properties. In order for a sunscreen to work efficiently, the inorganic sunscreen particles must be dispersed well in a personal care composition. However dispersion of ZnO particles in vanishing creams, where stearic acid is used as the oil phase, is always difficult due to aggregation of the particles. Ordinarily, the mixing temperature of the components of vanishing cream is 80° C. and mixing time is not controlled. As discussed below, ZnO reacts with stearic acid and forms zinc stearate. When pre-dispersed ZnO (available from Uniquema Spectraveil, Peterlee, England) is used, the zinc oxide particles form aggregates after mixing with stearic acid. Surface treated (with dimethicone/methicone) ZnO particles react even more rapidly with stearic acid. Much mechanical energy or surfactant is needed to separate the particles, which adds to the cost of the process of incroporating sunscreen in the compositions. Moreover, the particles tend to re-aggregate if mechanical energy is stopped or if the chemical system is changed, thereby resulting in poor dispersion stability.

Another problem with incorporation of ZnO in vanishing creams is the lack of chemical stability. ZnO reacts with fatty acid in the cream composition to form zinc stearate, which is a powder. Formation of the powder leads to loss of the desired sensory properties and of cream activity.

The reaction of ZnO with stearic acid to form zinc stearate is disclosed in U.S. Pat. No. 4,923,518 and 3,083,113; and Kruger, F. W. H., et al., “A DSC Study of Curative Interactions,” Journal of Applied Polymer Science, 42:2643-2649 (1991). However, controlling this reaction for ZnO stabilization in fatty acid environments, such as for cosmetics, had not been achieved.

Dispersion of ZnO in industrial applications is disclosed in U.S. Pat. No. 6,162,836. Dispersion of inorganic UV filters is disclosed in U.S. Pat. Nos. 5,916,544; 5,914,101 and 6,146,617.

The aggregation and lack of chemical stability are disadvantages that restrict the use therein of sufficient quantities of valuable ZnO sunscreen particles and add to the cost of incorporating sunscreen in cosmetic vanishing cream. However, emerging trends in skin care have required that vanishing cream formulations be adapted to incorporate sufficient sunscreen materials to protect from the strong sun, especially in the hot and sunny climates where vanishing creams are so popular. Therefore, it was clear that further work was necessary in order to enhance the performance of vanishing creams in their ability to incorporate sufficient sunscreen material at low cost.

Accordingly, there is a need to provide a process for incorporating ZnO into vanishing creams and vanishing cream cosmetic composition that not only maintains the unique sensory characteristics of vanishing cream but also enables a cost effective process to incorporate and disperse zinc oxide particles as an effective sunscreen.

### SUMMARY OF THE INVENTION

The shortcomings of the prior art are overcome with a process and composition that can provide the consumer-desired sensory properties of traditional vanishing creams, with the necessary amounts of inorganic sunscreen material. The present invention is based on the discovery that a reaction of ZnO in a fatty acid environment of a vanishing cream, controlled at less than 80° C., preferably about 60° C. to about 70° C., leads to the formation of a zinc stearate shell on ZnO nano-particles. The formation of the initial shell inhibits further reaction. Excellent dispersion is also achieved without any external addition of energy.

In one aspect, the present invention relates to a process of incorporating ZnO in a vanishing cream cosmetic composition comprised of solid asymmetric particles by:
Melting said solid asymmetric particles to form melted fatty acid;
Adding un-coated ZnO particles to the melted fatty acid to form a mixture of ZnO and fatty acid; preferably in an amount of about 1% to about 4%;
Heating said mixture to a temperature of less than about 80° C. for about 5 to about 10 minutes;
Cooling said to a temperature of about 50° C., thereby quenching any reaction between said ZnO and said fatty acid.

The present invention includes a cosmetic composition including:
(a) at least about 4% by weight of the composition of solid asymmetric particles;
(b) ZnO particles incorporated by the inventive process described above; preferably in an amount of about 1% to about 4%; and
(c) a cosmetically acceptable vehicle;
   and the composition has a normal stress of less than minus about 100 milli-Newtons. The solid asymmetric particles may be particles of a fatty acid containing from 12 to 22 carbon atoms and/or may optionally be crystalline.

The present invention also includes a method of controlling or preventing appearance tanning, by applying to the skin the inventive composition.

### DETAILED DESCRIPTION OF THE INVENTION

The present invention provides a process of dispersing ZnO in stearic acid or fatty acid environment of vanishing creams, which can achieve a high UV activity. The present invention also provides a vanishing cream composition having ZnO incorporated therein by the inventive process. The present invention is based on the theory (without wishing to be bound thereby) that, if a protective shell surrounds zinc oxide nano-particles in a cosmetic composition, it can prevent the undesirable chemical reaction between zinc oxide and the stearic acid in the composition and can work to disperse the ZnO in the composition. Applicants have discovered that the undesirable reaction can be controlled and, in fact, used to advantage, if the reaction time and temperature during the mixing of untreated zinc oxide nano-particles with stearic acid during the production of the composition are carefully controlled. Advantageously, the inventive process provides good dispersion of zinc oxide in the stearic acid phase.

The present invention is based on the discovery that a reaction of ZnO in a fatty acid environment of a vanishing cream, controlled at less than 80 deg. C. leads to the formation of a zinc stearate shell on ZnO nano-particles. The formation of the initial shell inhibits further reaction. Excellent dispersion is also achieved without any external addition of energy. Without being bound by theory, this is attributed to break up of the aggregates through the reaction.

The present invention relates to a process of dispersing zinc oxide nano-particles in vanishing cream compositions and vanishing creams incorporating thereby dispersed ZnO. In the course of preparing a vanishing cream composition, untreated zinc oxide nano-particles (for example, NANOX brand ZnO powder available from Rheox) are mixed with stearic acid and other components at a controlled temperature for a controlled time. The mixing temperatures are controlled at below about 80° C., preferably at about 60 to about 70° C. The reaction time is controlled at about 5 to about 10 min.

The inventive vanishing creams according to the present invention are cosmetic compositions that include:
(a) at least about 4% by weight of the composition of solid asymmetric particles;
(b) ZnO nano-particles incorporated by the inventive process; and
(c) a cosmetically acceptable vehicle;
and
wherein the composition has a normal stress of less than minus about 100 milli-Newtons.

Preferably, the asymetric particles are platelike, such that upon application to skin, the particles align under shear to produce the negative normal stress that is perceived as a dry and draggy feel on the skin surface. Preferred platelike asymetric particles are fatty acid crystals.

Untreated, or un-coated, ZnO nano-particles less than about 500 nm in mean particle diameter are included in the composition as a sunscreen, preferably less than about 200 nm. The untreated ZnO is added to the composition in an amount of about 0.1% to about 10% by weight of the composition, preferably about 1% to about 4% by weight of the composition.

The present invention also includes a method of controlling or preventing the appearance of tan, by applying to the skin the inventive composition.

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

As used herein, the term “comprising” means including, made up of, composed of, consisting and/or consisting essentially of.

The term “skin” as used herein includes the skin on the face, neck, chest, back, arms, hands, legs and scalp.

The term “solid” as used herein means that the material is not fluid at 25° C.

The term “fluid” as used herein means that the material is fluid at 25° C.

### Asymmetric Particles

The inventive compositions employ asymmetric solid particles, to impart a cream-like viscosity. Furthermore, by virtue of being asymmetric, the particles deliver high skin friction. Suitable solid particles include fatty acid crystals, mica, talc, clays and mixtures thereof. The preferred solid particles are selected from the fatty acid crystals wherein fatty acid contains from 12-22 carbon atoms, because they are inexpensive and the most aesthetically acceptable. The most preferred fatty acid is stearic acid. The inventive compositions contain at least 4% of the asymmetric solid particles, preferably at least 10%, more preferably from 10% to 25%, most preferably from 12% to 20% to obtain the best feel, appearance, and viscosity. The exact amount depends on the final composition and the nature of the other ingredients in the composition. The amount of the asymmetric solid particles, however, must be sufficient to impart the vanishing cream-like consistency to the composition, i.e. having the viscosity profiles at three defined shear rates as specified below under the defined measurement conditions.

Viscosity Profile of Vanishing Creams

In order to that the cosmetic compositions may be rubbed into skin easily, the inventive compositions have viscosity profiles of vanishing creams at three defined shear rates, as follows:

| | | |
|---|---|---|
| (1) | about 10 to about 75 Pascal- | at a shear rate of 1 reciprocal |
| | seconds (PaS) | second (s⁻¹), |
| (2) | about 2 to about 25 PaS | at a shear rate of 10 s⁻¹, and |
| (3) | about 0.6 to about 5 PaS | at a shear rate of 100 s⁻¹. |

Preferably the viscosity profile is:

| | |
|---|---|
| (1a) about 15 to about 60 PaS | at a shear rate of 1 s⁻¹, |
| (2a) about 2.5 to about 15 PaS | at a shear rate of 10 s⁻¹, and |
| (3a) about 0.8 to about 3 PaS | at a shear rate of 100 s⁻. |

More preferably, the viscosity profile is:

| | |
|---|---|
| (1b) about 20 to about 50 PaS | at a shear rate of 1 s⁻¹, |
| (2b) about 3 to about 7 PaS | at a shear rate of 10 s⁻¹, and |
| (3b) about 1 to about 2 PaS | at a shear rate of 100 s⁻¹. |

The viscosity referred to hereinabove is measured using a rheometer with a shearing force capability and normal force transducer (e.g. ARES, Advanced Rheometric Expansion System, from Rheometrics). Samples are compressed between parallel plates of a diameter 25 mm and gap (distance between two plates) of 100 microns. The measurements are made in a shear sweep mode with a shear rate range from 0 to 10,000 sec-1. Measurements are conducted at room temperature and pressure.

In the inventive compositions, the solid asymmetric particles are dispersed in water. Skin benefit agents, surfactants, sunscreens and other ingredients are employed in such amount as to provide a desired skin benefit and yet to not compromise the high skin friction of the inventive compositions.

Skin Friction/Normal Stress

The skin friction is measured by normal stress. Normal stress is the force exerted by the material in the axial direction during shearing flow. Normal stresses arise when the material (product) microstructure becomes anisotropic under flow. Negative normal stresses are generated by a decrease in bulk “volume” such as shear-induced ordering. Materials exhibiting this behavior are crystalline phases (e.g. fatty acids), rods, platelets, liquid crystals, and surfactant lamellar mesophases.

Measurement Procedure:

Normal force is measured using a rheometer with a shearing force capability and normal force transducer (e.g. ARES, Advanced Rheometric Expansion System, from Rheometrics). Samples are compressed between parallel plates of a diameter 25 mm and gap (distance between two plates) of 100 microns. The measurements are made in a shear sweep mode with a shear rate range from 0 to 10,000 sec-1. The normal stress value was calculated as the difference between the force at zero shear rate and force at highest shear rate. A negative difference of less than −100 milli-Newtons is correlated to products/materials with the draggy sensation (high skin friction). Measurement is conducted at room temperature (about 25° C.).

The inventive compositions have the normal stress of less than about minus 100 milli-Newtons; preferably less than minus 200 milli-Newtons; most preferably, in order to obtain the most desired high skin friction, less than about minus 100 milli-Newtons; e.g. from about minus 200 milli-Newtons to about minus 2000 milli-Newtons.

### ZnO Inorganic Sunscreen

To work effectively as sunscreens, inorganic sunscreen materials have to effectively attenuate UV light, by scattering and absorbing light in the 290-320 nm (UVB) and 320-400 nm (UVA) wavelength ranges. ZnO is a preferred inorganic sunscreen material because it absorbs and scatters light over a broad range of the UVB and UVA light spectrum.

The UV absorbance of the inorganic sunscreen powders is dependent on both the primary particle size and the agglomerate size. In accordance with the present invention, uncoated ZnO particles are dispersed in a cosmetic composition of the vanishing cream type. Uncoated ZnO is available under the NANOX brand from Rheox, Hightstowneee, N.J. ZnO nano-particles less than about 500 nm in mean particle diameter, in order to avoid gritty feel, are included in the composition as a sunscreen. Preferably, the ZnO nano-particles are less than about 200 nm in mean particle diameter, in order to better scatter light. More preferably, the ZnO nano-particles are about 100 nm to about 200 nm in mean particle diameter, in order for the particles to efficiently scatter light in the UV range.

Amount ZnO used in composition ranges at about 0.1% to about 10% by weight of the vanishing cream composition. Preferably, ZnO is used in an amount of about 1% to about 4%.

Other Optional Skin Benefit Materials and Cosmetic Adiuncts

Hydrophobically modified polymeric emulsifiers may be optionally present in the inventive compositions as a co-structurant, typically with a trade name, Pemulen TR® series, from 0.001 to 2% by weight, supplied by BF Goodrich Co., Cleveland, Ohio.

pH adjusting agents may be used to maintain the desired pH, if necessary. Preferred pH adjusting agents include inorganic or organic bases such as ammonium hydroxide, potassium hydroxide, sodium hydroxide and triethanolamine. Preferred pH adjusting agents also include inorganic acids such as HCI.

Emollient materials (fluid oils) selected from the groups of silicone oils or synthetic esters may be incorporated into the compositions of the present invention. The oils are employed in such amount as to not compromise the high skin friction of the inventive compositions. They may be present in a weight ratio to the solid particles, i.e. fatty acids, such that the composition exhibits a normal force value of less than −100 milli-Newton (mN) under the measurement condition defined previously. Weight ratio of emollient material to the solid particles may be less than 1.0, preferably, less than 0.5, most preferably, less than 0.2. Oily sunscreens, when used in the composition are considered to be emollient materials, and will be further discussed below.

Silicone oils may be included in the compositions as emollient materials. These are preferably chosen from cyclic or linear polydimethylsiloxanes containing from about 3 to about 9, preferably from about 4 to about 5, silicon atoms. Other silicone oils may be also included, such as polyalkyl siloxanes, polyalkylaryl siloxanes and polyether siloxane copolymers (e.g. dimethicone copolyol). The polyalkyl siloxanes useful herein include, for example, polydimethyl siloxanes with viscosities of from about 5 to about 100,000 centistokes at 25° C., preferably, polydimethyl siloxanes having viscosities from about 10 to about 400 centistokes at 25° C.

Suitable ester emollients include: esters of fatty acids or alcohols and hydrocarbons, preferably C8-C20 alkyl ester of fatty acids such as, isopropyl myristate, isopropyl palmitate, isostearyl palmitate, tridecyl salicylate, C12-15 octanoate and isopropyl stearate, or any mixtures thereof. The oils may be employed singly or in mixtures with one another.

The inventive compositions most preferably further include an ingredient selected from the group consisting of antioxidants, reducing agents, chelating agents, and mixtures thereof to improve the stability of the cosmetic cream. These ingredients provide an additional level of protection against oxidation of skin benefit agents in the cosmetic cream. Common examples of antioxidants, reducing agents and chelating agent for the present formulations can be found in the CTFA International Cosmetic Ingredient Dictionary 4^{th} Edition, The Cosmetic, Toiletry, and Fragrance Association, Inc., Washington, D.C., 1991.

Preferable reducing agents are sodium sulfite, sodium bisulfite, sodium metabisulfite, sodium thiosulfite or other thiols, such as thioglycerol, thiourea, thioglycolic acid, cysteine and the like. Preferable antioxidants are rac-6-hydroxy-2,5,7,8-tetra-methylchromane-2-carboxylic acid (trolox), propyl gallate, n-propyl trihydroxybenzoate, t-butyl hydroquinone and butylated hydroxytoluene (BHT), butylated hydroxyanisole (BHA), tocopheryl acetate, ascorbyl palmitate, hydroquinone, dibutyl hydroquinone and the like.

Suitable examples of chelating agents include, but are not limited to, EDTA, citric acid, tartaric acid, organo aminophosphonic acids and organo phosphonic acid components including certain of the commercially available Dequest™ compounds, marketed by Monsanto. Preferred is 1-hydroxyethylene, (1.1 -diphosphonic acid).

Organo aminophosphonic acid is an organic compound comprising of at least one phosphonic acid group, and at least one amino group. Suitable organo aminophosphonic acid components for use herein include the amino alkylene poly (alkylene phosphonic acids) and nitrilo trimethylene phosphonic acids. Examples of this type of organo aminophosphonic acid components include certain of the commercially available Dequest™ compounds, marketed by Monsanto.

Preferred are amino tri (methylene phosphonic acid) (Dequest 2006®), diethylene triamine penta (methylene phosphonic acid) and hexamethylene diamine tetra (methylene phosphonic acid).

Other suitable additional heavy metal ion sequestrants for use herein include nitrilotriacetic acid and polyaminocarboxylic acids such as ethylenediaminotetracetic acid, or ethylenetriamine pentacetic acid.

Still other suitable additional heavy metal ion sequestrants for use herein are iminodiacetic acid derivatives such as 2-hydroxyethyl diacetic acid or glyceryl imino diacetic acid.

Antioxidants are included in the inventive compositions in an amount of from 0.01 to 10%, preferably from 0.1 to 5%, most preferably from 0.2 to 4%. Reducing agents are included in the inventive compositions in an amount of from 0.01 to 10%, preferably from 0.1 to 5%, most preferably from 0.2 to 4%. Chelating agents are included in the inventive compositions in an amount of from 0.01 to 1%, preferably from 0.05 to 0.5%, most preferably from 0.05 to 0.3%.

Various other types of active ingredients may be present in cosmetic compositions of the present invention. Actives are defined as skin or hair benefit agents other than emollients and other than ingredients that merely improve the physical characteristics of the composition. Although not limited to this category, general examples include skin lightening agents, acidic skin benefit agents, sunscreens and tanning agents.

Sunscreens include those materials commonly employed to block ultraviolet light. Illustrative compounds are the derivatives of PABA, cinnamate and salicylate. For example, octyl methoxycinnamate and 2-hydroxy4-methoxy benzophenone (also known as oxybenzone) can be used. Octyl methoxycinnamate and 2-hydroxy4-methoxy benzophenone are commercially available under the trademarks, Parsol MCX and Benzophenone-3, respectively.

The exact amount of sunscreen employed in the emulsions can vary depending upon the degree of protection desired from the sun's UV radiation. However, the amount of sunscreens added should not compromise the high skin friction of the inventive compositions as defined previously for the emollient materials.

Another preferred optional ingredient is selected from essential fatty acids (EFAs), i.e., those fatty acids which are essential for the plasma membrane formation of all cells, in keratinocytes EFA deficiency makes cells hyperproliferative. Supplementation of EFA corrects this. EFAs also enhance lipid biosynthesis of epidermis and provide lipids for the barrier formation of the epidermis. The essential fatty acids are preferably chosen from linoleic acid, γ-linolenic acid, homo- γ-linolenic acid, columbinic acid, eicosa-(n-6,9,13)-trienoic acid, arachidonic acid, γ-linolenic acid, timnodonic acid, hexaenoic acid and mixtures thereof.

Other optional ingredients may include coloring agents, opacifiers and pigments (e.g. titanium dioxide, silica) and perfumes. Amounts of these materials may range anywhere from 0.001% up to 20% by weight of the composition.

### Process of Making Vanishing Cream with Inorganic Sunscreen

According to the present invention, un-treated ZnO particles are used in the stearic acid phase. The product is formulated, or mixed, at less than about 80° C., preferably less than about 70° C., more preferably at about 60° C., in the absence of silicone oil. Surprisingly, the product looks smooth and uniform. Electron microscopy images show that the ZnO particles are well dispersed. The reaction of ZnO with stearic acid is controlled to a certain limit, preferably conversion of about 5% to about 10%, by controlling the temperature and time of mixing. Differential scanning calorimetry (DSC) results show that there is only a little amount of zinc stearate formed. These are unexpected results. Without being bound by theory, this seems to be due to the formation of a protective shell of zinc stearate outside the zinc oxide particles, which prevents penetration of stearic acid into the ZnO particle and further reaction.

Reaction kinetics of the ZnO-stearic acid reaction at different temperatures was studied, and differential scanning calorimeter (DSC) was used to characterize and quantify the reaction. The mechanism was further verified by Raman microscopy imaging, AFM and FE-SEM. The reaction is exothermic and is sensitive to temperature. When the temperature exceeds about 80° C., the reaction proceeds rapidly. Without being bound by theory, this is thought to arise due to the collapse of the protective shell either due to structural changes or dissolution of zinc stearate. At temperatures below about 80° C., the reaction occurs by a diffusion controlled process and occurs within about 5 minutes of mixing at elevated temperature below about 80° C. There appears to be an optimal or critical temperature below which the reaction of ZnO and stearic acid should be controlled.

This process of incorporating ZnO in vanishing cream not only controls the reaction of ZnO with stearic acid, but also solves the problem of dispersing particles. Advantageously, the cost is much reduced compared with using pre-dispersed or surface-treated zinc oxide particles in the formulation.

Processes for making Vanishing Cream with Untreated ZnO in Oil Phase

1. Small Batch (100 g) with sonication (4% Nanox brand untreated ZnO, from Rheox)
Weigh beaker and stir bar, write down the number of total weight.
Weigh 77.29 g DI water in beaker
Weigh 0.97 g 50% KOH in the same beaker
Place beaker on hot plate (120° C.) and cover it with a piece of aluminum foil
Weigh 17.9 g Pristerene 4911 (stearic acid+palmitic acid) and place on hot plate until melted
Weigh 4 g Nanox powder and put in melted stearic acid. Stir with a spatula until the powder goes into the liquid and form a paste
Place the beaker with melted stearic acid and Nanox in a sonicator with water at about 70° C., and sonicate for 10-15 min
Pour the sonicated Nanox in stearic acid into water-KOH mixture, and stir at 800-900 RPM
After the emulsion is formed, lower the speed to 300 RPM, and turn off the heating for the hot plate.
When the temperature reaches 45° C., weigh the whole beaker with the emulsion and add some more DI water to make a 100 g formulation to compensate for the water loss during evaporation.

2. Big batch with ESCO-LABOR CH-4125 vacuum mixer
Check the vacuum system of the mixer to make sure it's sealed well
Set the temperature of the water bath to be 65° C.
Weigh 107.4 g stearic acid and put in the container of the vacuum mixer
After the stearic acid melts, weight 6 g Nanox and put in the stearic acid phase.
Mix the stearic acid and Nanox using the scraper in the container, operate at high speed.
Weigh 480.78 g DI water and 5.82 g 50% KOH. Heat up mixture to 60° C.
Pour the mixture of water and KOH into the container, where Nanox is well dispersed in stearic acid (no big blobs seen in the container).
Turn on the vacuum pump and keep the vacuum at −0.6bar. Turn on the scraper to mix the two phases.
Operate about 20 min to let the air bubbles go out. Turn off the heating water, and package the formulation when the temperature drops to lower that 45° C.
   Use of the Composition

The composition according to the invention is intended primarily as a product for topical application to human skin, especially as an agent for controlling tanning and/or for lightening or depigmenting the skin.

In use, a small quantity of the composition, for example from 1 to 5 ml, is applied to exposed areas of the skin, from a suitable container or applicator and, if necessary, it is then spread over and/or rubbed into the skin using the hand or fingers or a suitable device.

The composition may be used in a method of controlling or preventing appearance tanning, by applying to the skin the inventive composition.

Product Form and Packaging

The composition can be packaged in a suitable container to suit its viscosity and intended use by the consumer. For example, a composition can simply be stored in a non-deformable bottle or squeeze container, such as a tube or a lidded jar.

The invention accordingly also provides a closed container containing a cosmetically acceptable composition as herein defined.

The following specific examples further illustrate the invention, but the invention is not limited thereto.

### EXAMPLE 1

A vanishing cream formulation made according to the present invention is shown in the Table below.

TABLE 1

| Ingredient Trade and CTFA | | |
|---|---|---|
| Name | Phase | % |
| Stearic Acid (Pristerene 4911) | A | 17.90 |
| Cetyl alcohol | A | 0.53 |
| Silicone oil DC200 350cst | A | 0.80 |
| Parsol MCX | A | 1.25 |
| Parsol 1789 | A | 0.400 |
| Propylparaben | A | 0.10 |
| Methylparaben | A | 0.2 |
| Water | B | 65.61 |
| Glycerin USP | B | 1.00 |
| EDTA di Na | B | 0.04 |
| KOH(50%) | B | 0.968 |
| *WATER | C | BALANCE |
| Phenoxyethanol | C | 0.2 |
| Total | | 100.00 |

*Water Balanced to 100

The formulation presented in Table 1 is prepared in the following fashion. Phase A is heated at 75° C. Phase B is heated to 75° C. in a container separate from that of Phase A. Thereafter the phases are combined with mixing with heat being turned off. Phase C is heated to 62° C. and mixed into Phases A/B at 62° C. The mixture is cooled until 40° C. and then packed.

In the Examples that follow, ZnO of various types in incorporated in Phase A of the composition by various methods that are compared. In accordance with the present invention, untreated ZnO particles are preferred, available under the NANOX brand.

### EXAMPLE 2

This example illustrates an experimental method of studying the reaction of ZnO with pure stearic acid.

Zinc oxide undergoes a reaction with fatty acid to form zinc soap and water. The reaction occurs essentially between a weak acid and a basic metal oxide, and it can be expressed by the following formula (I):
ZnO+CₙH₂ₙ₊₁COOH→Zn(CₙH₂ₙ₊₁COO)₂+H₂O (I)

Reaction of pure stearic acid with zinc oxide was conducted to investigate the reaction conditions and to verify the characterization method for the reaction. 17.9 g pure stearic acid (Sigma-Aldrich, St Louis, Mo.) was added to a 100 ml beaker and the beaker was heated in a water bath at 75° C. After the stearic acid melted, 4 g Nanox ZnO (Rheox, Hightstown, N.J.) was added to the melt and stirred with a spatula until all ZnO powder was incorporated into the liquid. Then the beaker was placed in a sonicator (NEY Ultrasonik 57X, CA), held at 75° C. and the mixture was sonicated at high power for 5 min. The beaker was taken out of the water bath and cooled down at room temperature. A fraction of the sample was taken from the solidified mixture for DSC analysis.

Differential Scanning Calorimetry (DSC)

At room temperature, both ZnO and stearic acid are in solid state. The molecules in the solid state can not move to the interface of the two materials and the reaction is limited. In order for the reaction to take place, one or both of the reactants must be in liquid phase. This means the reaction temperature has to be equal to or higher than the melting point of stearic acid, which is 69-70° C. for pure stearic acid and 59° C. for Pristerene 4911 (See Example 3). Therefore, in this and the following Examples, the reaction temperature was kept at 75° C. when pure stearic acid was used and above 59° C. when Pristerene 4911 was used (in the following Example). Thus, both pure stearic acid and commercial Pristerene 4911 were used in the reaction to see the difference in reaction conditions and product. The reaction mechanism was investigated by studying the reaction kinetics at different temperatures.

To characterize the extent of the reaction for ZnO with stearic acid in a formulation, a TA Instruments (New Castle, Del.) DSC 2910 Differential Scanning Calorimeter was used to obtain the heat absorbed by those components undergoing phase transition when the temperature increases. The temperature for the phase transition is used to characterize the components and the area of the peak gives the absorbed heat for each phase transition.

The formation of zinc stearate during the heat up process was followed by DSC. The same sample was run by DSC twice with identical heating parameters in the same aluminum pan. The DSC graphs for the two heating cycles in this experiment showed two peaks at almost the same temperatures. A large peak shows up at about 70° C., and a small one at about 124° C. The first peak corresponds to the melting point of stearic acid (69-70° C) and the second one is close to that of the zinc stearate (120° C.) (Merck Index). Therefore, the peak at 70° C. was designated as stearic acid, and the one at 124° C. as zinc stearate.

A DSC graph shows the heat absorbed by the sample during a phase transition, measured in J/g and plotted versus temperature (deg. C.). The larger the amount of zinc stearate in the sample, the bigger the peak should be at about 120-124° C. The peak at 124° C. for the second run is larger than that for the first run, so it seems there is more zinc stearate formed when we run the sample for the second time. The reason for this is that when we ramp the temperature during the run, stearic acid in the sample melts and reacts with ZnO to form zinc stearate. However, when the scan rate is fast (10° C./min), it doesn't allow enough time for more stearic acid to react, so extent of reaction is still low and the second graph still shows a big stearic acid peak.

In order to quantify the amount of ZnO or stearic acid reacted, amounts of stearic acid and zinc stearate in the sample need to be calculated from the DSC graphs. Because the amount of sample varies, the heights of the peaks in different graphs cannot be compared. However, the area under each peak for one gram sample can be calculated by using the software provided with the DSC instrument “Universal analysis”. The amount of zinc stearate can be calculated using the protocol described hereinbelow. The percentage of reacted stearic acid for the first and second run was calculated to be 3.73 and 40.7% respectively.

The DSC graph for a mixture of Pristerene 4911 and Nanox ZnO, again, showed two peaks: one at 60° C., and the other at 114° C. The melting point of Pristerene 4911 is 54° C. The first peak appears at a higher temperature than the melting point of Pristerene 4911. Because the peak is very broad and the temperature at which the phase transition starts to occur is at a much lower temperature (about 50° C.), we designate the first peak as Pristerene 4911. We don't know the melting point of the mixture of zinc palmitate and zinc stearate. However, it is the only other possible component which can melt at 114° C., hence we designate the second peak to the reaction product. Comparing these results with the DSC graph for pure stearic acid showed that there is a decrease in peak temperature for both transitions for Pristerene 4911. The reason is that Pristerene 4911, a mixture of stearic acid and palmitic acid, has a lower melting point than pure stearic acid. A similar line of reasoning is expected to apply to the reaction product.

The heat absorbed for each phase transition per gram sample is calculated using the “Universal Analysis” software, available from TA Instruments, USA. The percentage of reacted fatty acid was calculated according to the Protocol described below and used to characterize the extent of reaction.

Protocol for Calculating Conversion of Zinc Oxide After Reaction with Stearic Acid

Molecular weight of the reaction species:
ZnO: 81.39, stearic acid (SA): 284.48, zinc stearate (ZnSA₂): 632.34

Fusion heat of the components undergoing phase transition:
stearic acid: 199.7 J/g
zinc stearate: 169 J/g

Reaction mass balance:

ZnO + 2SA → ZnSA₂ + H₂O

| | | | |
|---|---|---|---|
| 81.39 | 568.96 | 632.34 | 18 |
| 2.56 | 17.9 | 19.9 | |

Original materials: 4 g ZnO+17.9 g stearic acid

Amount of stearic acid and zinc stearate in the DSC sample was calculated from the DSC graphs (NOTE: “*” denotes multiplication):

Heat absorbed by phase transition of stearic acid (from area of DSC peak): a

Heat absorbed by phase transition of zinc stearate (from area of DSC peak): b

Amount of stearic acid (SA) left from the reaction: A=a/199.7

| | |
|---|---|
| Amount of ZnSA formed: | b/169 |
| Amount of SA reacted: | B = b/169/632.34 * 568.96 + 5.315E−3 * b |

Percentage of reacted stearic acid: B/(A+B)

Percentage of reacted ZnO: B/(A+B)*2.56/4

### EXAMPLE 3

### Reaction kinetics of ZnO with Pristerene 4911

This example illustrates a study of the reaction kinetics of Nanox zinc oxide with commercial stearic acid used in the formulation of Example 1, Pristerene 4911 brand (45% stearic acid and 55% palmitic acid, CK Witco Corporation, Memphis, Tenn.). The temperature and time required for ZnO to react with Pristerene 4911 were investigated by observing the percentage of reactant over time at different temperatures.

17.9 g Pristerene 4911 was placed in a 50 ml beaker immersed in a temperature controlled water bath. The temperature was maintained at 2-3 degrees above the desired reaction temperature. After the Pristerene melted, 4 grams ZnO (Nanox) powder was added to it with continuous stirring at 40 RPM. About 200 micro-I sample was taken from the beaker at designated time intervals and placed on a glass slide to cool down and solidify. DSC experiments as described in Example 2 were conducted on these samples to characterize the extent of reaction, and the conversion was calculated according to the Protocol of Example 2. Because there is excess ZnO stoichemically in the mixture, the extent of reaction is calculated by percentage of reacted Pristerene 4911. Conversion of ZnO can be easily calculated from that of Pristerene 4911.

The Table below summarizes the data demonstrating reaction kinetics for Pristerene 4911 with Nanox ZnO at different temperatures.

TABLE 2
Percentage of Nanox ZnO reaction with Pristerene 4911 at
different reaction temperatures and reaction times

| time | | | | |
|---|---|---|---|---|
| (min) | 60° C. | 70° C. | 80° C. | 85° C. |
| 2 | 3 | 2.72 | 4.1 | 3.02 |
| 5 | 2.67 | 4.28 | 4.9 | 6.67 |
| 10 | 2.84 | 5.69 | 6.42 | 9.6 |
| 20 | 1.75 | 6.9 | 4.79 | 95 |
| 40 | 1.54 | 6.5 | 52 | |
| 60 | 2 | 10.3 | 73.4 | |
| 120 | 2.17 | 8.24 | | |

The data in the Table above shows that at temperatures lower than 80° C., there was a small percentage of reaction shortly after ZnO and Pristerene 4911 were mixed together. After that the kinetics curve levels off. Even two hours after the mixing, the percentage of reaction hardly changed. However, if the reaction temperature is equal or higher than 80° C., the percentage of reaction increases significantly with time. It was also observed in this experiment for the high temperature reaction was that the temperature in the beaker could not be kept constant during the reaction. The temperature increased so fast that before it was cooled down by the water bath, the reaction has finished. The temperatures reached were as high as 120° C. This demonstrates that the reaction is exothermic.

The ZnO-stearic acid reaction is a solid-liquid heterogeneous reaction. Only the surface of the solid or particle can be in contact and reacts with the liquid. The reaction product zinc stearate is not soluble in Pristerene 4911, hence it probably deposits on the surface of the ZnO particles and eventually forms a shell. When the shell of the product is dense or very thick, Pristerene 4911 may not able to penetrate the layer. The reaction may stop at any time point. At the beginning, the reaction product, i.e., zinc stearate, forms a shell outside the reactant particle, which seems to be the case for the reactions at 60° C. and 70° C. The thickness of the shell, as a percentage of the radius may be measure by Raman Micro-spectroscopy, and is discussed in more detail hereinbelow. If the liquid can penetrate the shell and reach the reactant surface, reaction proceeds and the product shell thickens until all solid reactant is consumed. When the reaction temperature is higher than 80° C., the solubility of zinc stearate in stearic acid is high enough that some of the zinc stearate coating dissolves into the liquid and the “protective layer” is damaged. Or possibly the zinc stearate melts and peels off from the ZnO particle. When this happens, stearic acid can diffuse to the surface of the zinc oxide and further reaction occurs.

The kinetics of the reaction can be described as a process of reaction-controlled or diffusion-controlled or a combination of the two. For a reaction-controlled process, the reaction rate is much lower than the diffusion rate. Because diffusion can be deemed negligible in a reaction controlled process, the reaction kinetics are the same as that of a homogeneous reaction, in which the reaction only stops when one or both of the reactants are exhausted. For this reaction, at lower temperatures, the kinetics data show that the reaction occurred fast in the first few minutes, then it slowed down and stopped even if the extent of reaction is very low (5-10%). We can deduce that the reaction is a diffusion-controlled process.

Verification by Raman Micro-Spectroscopy

The existence of the zinc stearate layer outside the ZnO particle was verified by Raman micro-spectroscopy. The sample examined was a mixture of 1-5 micro-meter diameter size ZnO particle reacted (mean diameter of about 4 micro-m) at 75° C. with stearic acid for 10 min.

From analysis of the Raman mapping images, it is clear that there is a ring of zinc stearate outside of zinc oxide, as evident from the different colors that appear in the images. The thickness of the ring particle is about 0.6 micro-m.

The thickness of the zinc stearate layer may be estimated from the percentage of reaction, density of ZnO and zinc stearate. Because of the huge difference in density and molecular weight between zinc stearate and zinc oxide (density 5.67 g/ml for ZnO vs. 1.095 g/ml for zinc stearate, and molecular weight 632.34 for zinc stearate vs. 81.39 for ZnO), a small volume of ZnO (1 ml) can form a large volume of zinc stearate (40.23 ml) after the reaction. For 5% reaction of ZnO, the thickness of the zinc stearate layer is 31% of the radius of the particle. For such a thick shell, the diffusion time for stearic acid into the particle is very long, which is the reason why this reaction is a diffusion-controlled process.

### EXAMPLE 4

This example demonstrates the UV absorbance of the formulation of Example 1. The performance of a sunscreen formulation is determined by how well it can attenuate UV light.

The UV absorbance of the product films at different wavelengths can be obtained by an SPF 290 machine, which is a modified UV spectrophotometer with an integrating sphere, available Optometrics LLC, Ayer Mass., USA. In order to compare the efficacy of different formulations, we need to compare UV absorbance for a dry film of the same thickness. To summarize the characterization protocol, due to uncontrollable thickness of the product film, the UV absorbance of a formulation in a 20 micro-m UV cuvette was first measured. Separately, the UV absorbance for a wet and dry draw-down film on a quartz plate were measured. Assuming a 20 micro-m thick wet drawn-down film has the same UV absorbance as a 20 micro-m thick film in the UV cuvette, the dry film for a product film with same wet thickness can be calculated.

Sunscreen formulations that exhibit good product characteristics—i.e. uniform cream with small particles, were selected and their UV attenuation properties were identified and compared. Selected vanishing cream formulations of Example 1 were made in a 1 kg scale ESCO-LABOR CH-4125 vacuum mixer and are summarized in the Table below.

TABLE 3
Vanishing Cream With Sunscreen formulations

| ZnO raw material | Processing | Formulation characteristics |
|---|---|---|
| 1% Nanox | In oil phase | Homogeneous and smooth |
| 2% Nanox | In oil phase | Homogeneous and smooth |
| 4% Nanox | In oil phase | Homogeneous and smooth |
| 4% Nanox | In water phase at pH 14 | Some aggregates |
| 4% Spectraveil | Post-added after | Small visible white pockets |
| | emulsification | |

The term MPF reflects UV attenuation by the formulation film. The data show that ZnO has a broad UV attenuation spectrum. It covers wavelength ranging from 290 to 370 nm, almost the whole UVB and UVA region. There is a significant decrease in UV attenuation when the film is dried. The UV attenuation curves as in FIG. 8 were used to obtain a MPF value at the wavelength of either 320 nm or 360 nm for the wet and dry vanishing cream films to calculate the MPF for a 20 μm thick film described above. Because ZnO in a formulation works more as a UVA sunscreen than a UVB sunscreen, the results for MPF at 360 nm for the above formulations are summarized in the Table below. The MPF numbers for a 20 μm dry film are used to compare the efficacy of different formulations.

TABLE 4
Comparison of the UV attenuation of different
formulations at 360 nm

| | MPF in 20 μm | MPF of | MPF of | MPF for 20 μm |
|---|---|---|---|---|
| Formulation | cuvette | wet film | dry film | dry film |
| 1% Nanox in oil | 5.83 | 5.89 | 4.88 | 4.83 |
| phase | | | | |
| 2% Nanox in oil | 32 | 13.3 | 4.3 | 10.35 |
| phase | | | | |
| 4% ZnO in | 38 | 51.4 | 4.98 | 13.58 |
| Spectraveil, | | | | |
| post-added | | | | |
| 4% Nanox in | 46 | 225 | 26.5 | 5.42 |
| water phase | | | | |
| added at pH 14 | | | | |

For these formulations, it seems the one with Nanox in oil phase formulation has better UVA attenuation than the other ones. Even with 2% Nanox, the MPF of the formulation is 10.35, which is comparable with 4% Spectraveil, which has visible aggregates in the formulation. The reason for a better UV attenuation with the Nanox in oil formulation is due to better dispersion of the ZnO particles.

### EXAMPLE 5

This example shows a simplified formulation set forth in the Table below. 4% Nanox ZnO in the stearic acid oil phase is added to the formulation of Table 2 according to the present invention.

TABLE 5

| Ingredient Trade and | | |
|---|---|---|
| CTFA Name | Phase | % |
| Stearic acid | A | 17.90 |
| Water | B | 81.13 |
| KOH(50%) | B | 0.97 |
| Total | | 100.00 |

### EXAMPLE 6

Structure of the product film affects its optical and sensory properties. Here structure includes both size and distribution of particles and emulsion droplets. To look at the microstructure of the product, field emission scanning electron microscopy (FE-SEM) was used in this Example. The results for formulations of Table 7 in Example 5 with 4% Tri-K 500SA brand TiO₂ and 4% Nanox brand ZnO incorporated in stearic acid oil phase are summarized as follows.

The formulation according to Example 5 was placed on a piece of aluminum sheet, and kept in a humidified environment. The imaging was conducted at low voltage, on a XL30 ESEM FEG, (FEI company, Swampscott, Mass.), so there was less sample damage by the electron than using conventional SEM.

For FE-SEM, the samples were not dried, so the stearic acid flakes were not seen. The particles of TiO₂ and ZnO were seen on the images. On the image for 4% TiO₂, big aggregates of about 1-2 micro-m were seen. However, for ZnO the particles appeared well distributed and the size of each particle was about 60 nm, which is close to the primary particle size of Nanox.

Clearly, the FE-SEM of this Example showed that the ZnO particles are much better dispersed than the TiO₂ particles in the sample.

### EXAMPLE 7

This example demonstrates a comparison of different ZnO raw materials and incorporation thereof into vanishing cream compositions by different processes.

Reaction in Different Formulations

There are different process options for incorporating ZnO into a vanishing cream formulation. ZnO can be added:
(1) in the Pristerene 4911 oil phase,
(2) in the water phase, and
(3) post added after the emulsion is formed.

In the water phase, ZnO can be incorporated:
(a) in DI water first at pH 6.5, or
(b) after KOH is added to water phase at pH 14.

Choices of ZnO include un-coated, hydrophobically coated, and pre-dispersed.

Different ways of incorporating ZnO raw materials in vanishing cream formulation of Example 5 (simplified vanishing cream formulation) and including 4% ZnO by weight were tested. The 100 g scale formulation method (small batch, as described herein above) was used in this study. The Table below gives a summary of the formulations and comments on observations made during the experiments.

TABLE 6
Comparison of different simplified vanishing cream
formulations with ZnO

| | | Formulation |
|---|---|---|
| ZnO raw material | Processing | characteristics |
| Rheox Nanox | In oil phase mixed by stirring | Some white |
| (un-coated) | | pockets |
| | In oil phase, mixed by sonication | Smooth and |
| | | homogeneous |
| | In water phase at pH 6.5 | With some |
| | | particles |
| | In water phase at pH 14 after adding | With some |
| | KOH | particles |
| | AFTER EMULSIFICATION | Powder |
| | 3% pre-dispersed in water by milling, | |
| | 1% in oil | |
| Tri-K 505S | In oil phase | Powder |
| (methicone | | |
| coated) | | |
| | Pre-dispersed in 5% silicone oil and | Powder |
| | added to oil phase | |
| | Pre-dispersed in 5% silicone oil and | Solid |
| | post added to formulation | |
| Uniqema | In oil phase | With some |
| Spectraveil | | particles |
| (dispersed in IPM | | |
| with poly- | | |
| hydroxystearic | | |
| acid) | | |
| | Post added after emulsification | With some |
| | | particles |
| Sunsmart HP1 | In oil phase | Half powder |
| (dimethicone | | |
| coated) | | |

Among the formulations of the Table above, some are creams with noticeable solid particles or aggregates (ZnO in water phase or post-added, Spectraveil, which is pre-dispersed ZnO in IPM, in oil phase or post-added). The existence of the solid particles causes poor sensory qualities and decreases the sunscreen efficacy. Others lose their cream characteristics and become powder or solid, which is undesirable for a product:
pre-dispersed ZnO placed in water phase or post-added,
hydrophobically coated ZnO incorporated in Pristerene 4911 oil phase,
pre-dispersed hydrophobic ZnO in silicone oil added in Pristerene 4911 oil or post added).
   The only formulation that is a uniform cream and with no noticeable particles is the one with 4% ZnO in Pristerene 4911 oil phase.

DSC was used to characterize the reaction in these formulations. The percentage of reacted Pristerene 4911 was calculated by the protocol in Example 2. The extent of the reaction greatly affects the product characteristics. When a small amount of reaction occurs (less than 10%), the formulation stays in its cream form. If a significant amount of the Pristerene 4911 reacts, the formulation turns into a powder or a solid. This can be explained by the significant solid volume increase when zinc salt of Pristerene 4911 is formed.

The formulations were also examined under an optical microscope to assess the size of the solid particles or aggregates formed in the formulations. In powder-like formulations, the size of the particles is very small, because it is a powder of zinc stearate. In cream-like formulations, the particles are aggregates of ZnO.

### EXAMPLE 8

This example demonstrates the chemical stability of the ZnO vanishing cream compositions of the present invention upon storage. From the Examples above, by comparing the physical characteristics and optical properties of the different formulations, the best formulations right after they are made were determined. However, a product in the market has to be stored in a warehouse and placed on the shelf of a store for several months, even a few years. Thus chemical or storage stability of the formulation is an important consideration, as well. Because the DSC graphs of the formulations can tell the extent of the reaction, which is linked to the appearance and optical properties of the formulations, it was again used to characterize the stability of formulations in this Example.

Based on the Experiments above, Nanox ZnO in Pristerene 4911 oil phase and Spectraveil post-added seem to be the formulations that gave the best results and best UV attenuation. Stability of these formulations with varied neutralization and percentage of IPM solvent was studied. The formulations tested were 4% Nanox ZnO in Pristerene 4911 oil phase with neutralization at 10%, 12.9% (standard), 20% and 30%, and Spectraveil post-added with 2.5% and 5% IPM. The formulations were stored in a 50° C. oven for a long time and samples were taken from the formulations for DSC experiments to test the stability of the formulations. Percentage of Pristerene 4911 reacted at different storage time was calculated according to the protocol in Example 2 using the DSC graphs. And the percentage of reaction is tabulated vs. time for different formulations in the Table below.

TABLE 7
Stability of different Vanishing Cream formulations placed in
50° C. oven and 4% Nanox in oil phase formulation
stored at room temperature.

| Time | | | | | | | |
|---|---|---|---|---|---|---|---|
| from | | % | | | | | |
| making | % | reac- | % | % | % | % | % |
| formu- | reaction | tion | reaction | reaction | reaction | reaction | reaction |
| lation | 12.9% | 2.5% | 5% | 10% | 20% | 30% | Room |
| day | Neutral | IPM | IPM | Neutral | Neutral | Neutral | temp. |
| 1 | 0 | 0 | 0 | 3 | 0 | 0 | 3 |
| 2 | 10 | 0.5 | 0.5 | 17 | 10 | 1 | |
| 4 | 16 | 0.5 | 0.5 | 19 | 13.6 | 2 | |
| 7 | 21.8 | 0.5 | 1 | 25 | 18.2 | 3 | |
| 14 | 24 | 0.5 | 1 | 28.3 | 33.3 | 44.6 | |
| 29 | 28.3 | 1 | 1 | 32.6 | 100 | 50 | |
| 60 | 100 | 1 | 1 | 100 | | 100 | |
| 90 | | | | | | | 10 |

From the stability data in the Table above, it seems that at 50° C., the formulation is stable when ZnO is in aggregate form, as when Spectraveil was used with addition of 2.5% and 5% IPM. However, when ZnO is added to stearic oil phase, the formulations held for only one month. Two months later, the reaction was completed, and the formulations fell apart. To slow down the reaction, we may optimize the thickness of the zinc stearate layer by a better control of the reaction time and temperature.

From the data, temperature appears to affect the stability of the formulations. At room temperature, the formulation is much more stable. DSC graph for a four-month old formulation with 4% Nanox placed in oil phase showed less than 10% reaction was completed.

It will be understood that the embodiments described herein are merely exemplary and that a person skilled in the art may make many variations and modifications without departing from the spirit and scope of the invention. Thus, all such variations and modifications are intended to be included within the scope of the invention as defined in the appended claims. Throughout this application, various publications have been cited. The entireties of each of these publications are hereby incorporated by reference herein.

1. A process for incorporating ZnO particles in a cosmetic composition comprised of solid asymmetric particles, comprising:
Melting said solid asymmetric particles to form melted fatty acid;
Adding un-coated ZnO particles to said melted fatty acid to form a mixture of ZnO and fatty acid;
Heating said mixture to a temperature of less than about 80° C. for about 5 to about 10 minutes;
Cooling said to a temperature of about 50° C., thereby quenching any reaction between said ZnO and said fatty acid.

2. The process according to claim 1, wherein said ZnO is added in an amount of about 0.1% to about 10% by weight of said cosmetic composition.

3. The process according to claim 1, wherein said ZnO is added in an amount of about 1% to about 4% by weight of said cosmetic composition.

4. The process according to claim 1, wherein said ZnO has a particle size diameter of less than about 500 nm.

5. The process according to claim 1, wherein said ZnO has a particle size diameter of less than about 200 nm.

6. The process according to claim 1, wherein said solid asymmetric particles are comprised of stearic acid.

7. The process of claim 1, wherein said solid asymmetric particles comprise about 10% to about 25% by weight of said composition.

8. The process of claim 1, wherein said heating temperature is about 60° C. to about 70° C.

9. The process of claim 1, wherein said ZnO reacts with said fatty acid and said reaction is controlled to a conversion of about 5% to about 10% of said ZnO.

10. A cosmetic composition comprising:
(a) at least about 4% by weight of the composition of solid asymmetric particles;
(b) zinc oxide particles incorporated by the process according to claim 1; and
(c) a cosmetically acceptable vehicle; and
wherein the composition has a normal stress of less than minus about 100 milli-Newtons.

11. The composition of claim 10, further comprising nonionic emulsifier selected from the group consisting of sorbitan ester, ethoxylated sorbitan ester, ethoxylated alky ether, ethoxylated fatty acid ether, fatty alcohol, ethoxylated fatty alcohol and ester of glycerin and fatty acid.

12. The composition of claim 10, further comprising anionic emulsifier in a ratio of said anionic emulsifier to said nonionic emulsifer is in the range of about 1:0.0000001 to about 1:9.

13. The composition of claim 12, wherein said anionic emulsifier remains anionic at low pH.

14. The composition of claim 12, wherein said anionic emulsifier is present in an amount of about 0.1% to about 10% by weight of said composition.

15. The composition of claim 12, wherein a ratio of said anionic emulsifier to said nonionic emulsifier is set such that said composition exhibits viscosity profiles of 10 to 75 Pas, 2 to 25 Pas and 0.5 to 6 Pas at the shear rates of 1 s⁻¹, 10 s⁻¹ and 100 s^{-,} respectively; wherein the composition has a normal stress of less than minus about 100 milli-Newtons.

16. The composition of claim 10, wherein said solid asymmetric particles comprise about 12% to about 20% by weight of said composition.

17. The composition of claim 10, wherein the solid asymmetric particles are crystalline.

18. The composition of claim 10, wherein the viscosity of the composition is 10 to 75 Pas, 2 to 25 Pas and 0.5 to 6 Pas at the shear rates of 1 s⁻¹, 10 s⁻¹ and 100 s⁻, respectively.

19. The composition of claim 10, wherein said solid asymmetric particles are particles of a fatty acid containing from 12 to 22 carbon atoms.

20. A method of controlling or preventing appearance tanning by applying to skin the composition of claim 10.
